(19) United States
(12) Patent Application Publication
(10) Pub. No.: US 2005/0107296 A1
(43) Pub. Date: May 19, 2005

(54) Isolated peptide of the hepatocyte growth factor and its variants, preparation method and therapeutic use as anti-angiogenic agents

(75) Inventor: Tatiana Merkoulova-Rainon, Paris Cedex 10 (FR)

Correspondence Address: OSTROLENK FABER GERB & SOFFEN, 1180 AVENUE OF THE AMERICAS, NEW YORK, NY, 100368403, US

(73) Assignee: Institut des Vaisseaux et du Sang

(21) Appl. No.: 10/835,986

(22) Filed: Apr. 29, 2004

Related U.S. Application Data
(63) Continuation of application No. PCT/FR02/03739, filed on Oct. 30, 2002.

(30) Foreign Application Priority Data
Oct. 31, 2001 (FR) 01 14 146

(51) Int. Cl. A61K 38/18

(52) U.S. Cl. 514/12

(57) ABSTRACT

The present invention concerns a peptide substance selected from the group consisting of: (i) peptide N derived from the HGF α-chain and having the amino acid sequence represented by SEQ ID No.: 2, and (ii) its peptide variants derived from said peptide N sequence by deletion, substitution or addition of one or more amino acids, said peptide N and its variants having an anti-angiogenic activity in accordance with a mechanism different from competitive binding of HGF to the HGF-R receptor. It also concerns the therapeutic use and the method for preparing said peptide substance, and (i) the DNA fragment which expresses it, the plasmid containing said DNA and the bacterium containing said plasmid.

### CROSS REFERENCE TO RELATED APPLICATIONS

This is a continuation of International Application PCT/FR02/03739, filed Oct. 30, 2002, published in the French language which claims priority of French Application No. 01 14 146 filed Oct. 31, 2001.

### FIELD OF THE INVENTION

The present invention relates, as novel industrial products, to an isolated peptide (herein referred to as N) of hepatocyte growth factor (HGF) and its variants. It also concerns the method for preparing them and their therapeutic use as anti-angiogenic agents intervening as inhibitors of angiogenesis.

The abbreviations and acronyms used herein are fully defined later.

### PRIOR ART

HGF is currently recognized as being a pleiotropic factor which possesses quite a broad specificity with respect to many cell targets. In particular, it plays an important role of mediator in the mesenchymal-epithelial/endothelial interactions which participate in complex biological processes such as embryogenesis, tissue regeneration and tumorogenesis [see K. Matsumoto et al., Ciba Found. Symp., 1997; 212: 198-211]. It is also known that HGF is a potent activator of angiogenesis [see F. Bussolino et al., J. Cell Biol., 1992; 119: 624-641], the in vivo efficacy of which could be greater than that of VEGF [see E. van Belle et al., Circulation, 1998; 97: 381-390].

Structurally speaking, HGF is a peptide related to the enzymes of the blood coagulation cascade. Mature HGF is a peptide composed of two chains (or subunits) : the α- and β-chains. In native HGF (see FIG. 1 hereinafter), the portion of the amino acid sequence (aa 1-478) which provides the α-chain comprises, from its N-terminal end to its C-terminal end:
(i) a peptide L comprising 31 amino acid residues (aa 1-31), which (i) is probably of no interest in pharmacological terms and (ii) is involved as a signal peptide,
(ii) a domain N containing a “hairpin” loop, having 96 amino acid residues (aa 32-127) and comprising four Cys residues (the peptide N is that which is useful according to the present invention), and
(iii) four kringle domains (called K1, K2, K3 and K4) each comprising 6 or 7 Cys residues, K1 comprising 79 amino acid residues (aa 128-206), K2 comprising 78 amino acid residues (aa 211-288) , K3 comprising 79 amino acid residues (aa 305-383) and K4 comprising 79 amino acid residues (aa 391-469).

The α-chain of HGF represents the main site of interaction of HGF with its receptor which is expressed by the c-Met proto-oncogene and which (after binding of HGF to its receptor) has tyrosine kinase enzymatic activity. In the text which follows, this receptor is referred to as HGF-R.

The β-chain, which exhibits homology with the catalytic domain of serine proteases, does not appear to bind to the HGF-R receptor, but is essential for the autophosphorylation of said receptor and, consequently, for its interaction with certain agents (“effectors”) involved in the intracellular signaling.

Lacking the β-chain, the peptide consisting of the α-chain, which binds to the HGF-R receptor, is an HGF antagonist [see K. Date et al., FEBS Letters, 1997; 420: 1-6] and acts as an inhibitor of the biological responses of HGF, in particular the angiogenic response of this factor [see K. Kuba et al., Cancer Res., 2000; 60: 6737-6743], according to a competition mechanism.

It is known, in particular from WO-A-99/55361, that the HGF α-chain has been sequenced. More precisely, it is known that the peptide referred to as NK4, which begins with a pyroglutamate group and comprises the amino acids of said α-chain (i.e. aa PyrGlu³² to Val⁴⁷⁸ of native HGF), has antitumor effects [see EP-A-0890361] and anti-angiogenic effects which manifest themselves through inhibition of neovascularization [see the publication by K. Kuba et al. and application WO-A-99/55361, mentioned above].

Briefly, the mechanisms known to date are as follows:
(A) by cleavage of peptide L, native HGF gives (or is “activated” to) mature HGF;
(B) mature HGF binds, via its α-chain, to its HGF-R receptor;
(C) as a result of the binding of mature HGF to HGF-R, the enzyme activity of HGF-R, which is of the tyrosine kinase type, manifests itself; this involves an “activation” by virtue of which the enzyme contained in the intracellular domain of the HGF-R protein becomes capable of exerting its tyrosine kinase effect;
(D) when it is used, peptide NK4, which binds to HGF-R without said receptor exerting its tyrosine kinase enzyme activity, acts as a competitive inhibitor of HGF.

### ORIGIN OF THE INVENTION

Since the K1, K2, K3 and K4 domains of the HGF α-chain are quite similar to the anti-angiogenic kringles of certain other proteins (such as angiostatin and the second kringle of prothrombin), the question was posed, during the development of the present invention, of whether (1) the kringles K1, K2, K3 and/or K4 of the HGF α-chain have anti-angiogenic activity and whether (2) the mechanism of the possible anti-angiogenic activity of one or more of these kringles lies in the competitive binding of K1, K2, K3 and/or K4 to the HGF-R receptor.

After having isolated and tested peptides N, K1, K2, K3 and K4 of the HGF α-chain, we have just found, surprisingly, that peptide N, which is substantially included in the sequence of NK4 [N differs from the sequence of NK4 by the nature of the first amino acid, Gln¹ replacing PyrGlu¹ (i.e. at position 32 of native HGF)], has anti-angiogenic properties which manifest themselves according to a mechanism different from that of the kringles of NK4.

Briefly, as illustrated hereinafter, it has been observed according to the invention that:
N acts as an inhibitor of HGF according to a mechanism different from the competitive binding with HGF to the HGF-R receptor; according to the results of the applicant's assays, N does not substantially bind to HGF-R, unlike NK4; there is every reason to think that N acts as an inhibitor of HGF from the moment that it interferes with the interaction of HGF with GAGs by inhibiting HGF/GAG binding; and

K1, unlike K2, K3 and K4, is, to the applicant's current knowledge, the only kringle of HGF to act effectively by competition with HGF for binding to the HGF-R receptor.

Subsidiarily, peptide N, which comprises the hairpin of NK4 and is derived from the HGF α-chain, contains in its sequence amino acids 2-96 of NK4 (i.e. Arg³³-Asn¹²⁷ of native HGF) but differs from NK4 by the first amino acid (i.e. Gln³² instead of the pyroglutamate group PyrGlu³² of native-HGF according to EP-A-0890361 and WO-A-99/55361, mentioned above).

### AIM OF THE INVENTION

According to the invention, it is proposed to provide (a) a novel substance which has anti-angiogenic properties, (b) a method for preparing it and (c) a therapeutic use of this substance.

### SUBJECT OF THE INVENTION

According to the invention, a novel peptide substance is therefore advocated, which is characterized in that it is selected from the group consisting of:
(i) peptide N derived from the HGF α-chain and having the amino acid sequence represented by SEQ ID No.: 2, and
(ii) its peptide variants derived from said peptide N sequence by deletion, substitution or addition of one or more amino acids,
   said peptide N and its variants having an anti-angiogenic activity in accordance with a mechanism different from competitive binding of HGF to the HGF-R receptor.

According to another aspect of the invention, a therapeutic composition is advocated, which is characterized in that it contains, in combination with a pharmaceutically acceptable excipient, a therapeutically effective amount of a peptide substance selected from the group consisting of:
(i) peptide N derived from the HGF α-chain and having the amino acid sequence represented by SEQ ID No.: 2, and
(ii) its peptide variants derived from said peptide N sequence by deletion, substitution or addition of one or more amino acids,
   for combating angiogenesis-related disorders.

The use of said anti-angiogenic peptide substance with respect to said angiogenesis-related disorders is also advocated.

Finally, the DNA fragment encoding peptide N or its abovementioned variants, the plasmid containing said DNA fragment and the bacterium containing said plasmid are also advocated as novel industrial products.

### BRIEF DESCRIPTION OF THE DRAWINGS

In the attached drawings,

FIG. 1 represents diagrammatically the structure of native HGF with the five main domains of the α-chain (N, K1, K2, K3 and K4), the fragment PD representing the β-chain;

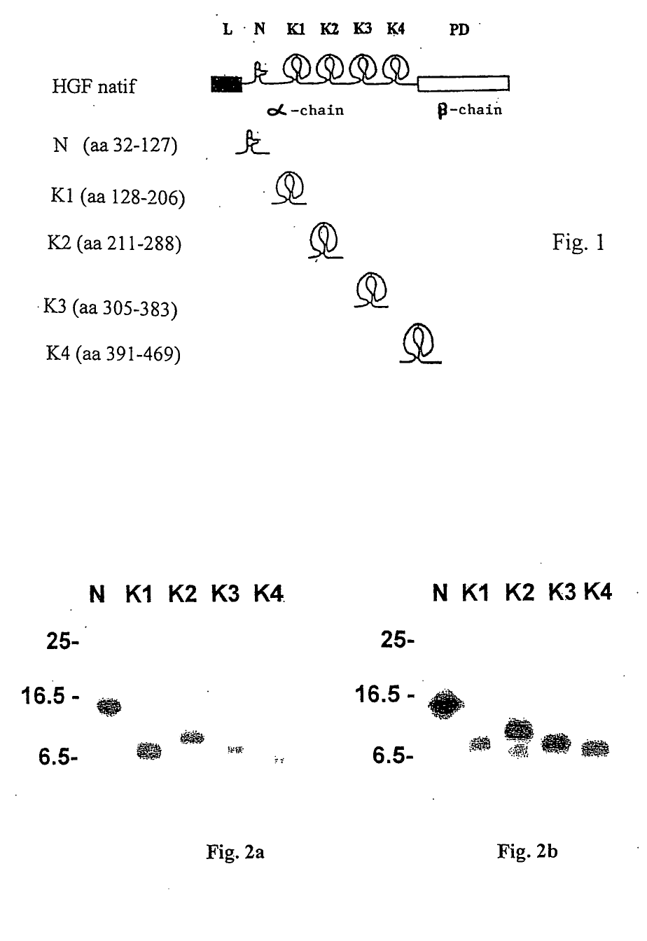

FIGS. 2a and 2b show the degree of purification of isolated N, K1, K2, K3 and K4 peptides according to coomassie blue staining (FIG. 2a) and Western blotting with an anti(HGF) antibody (FIG. 2b);

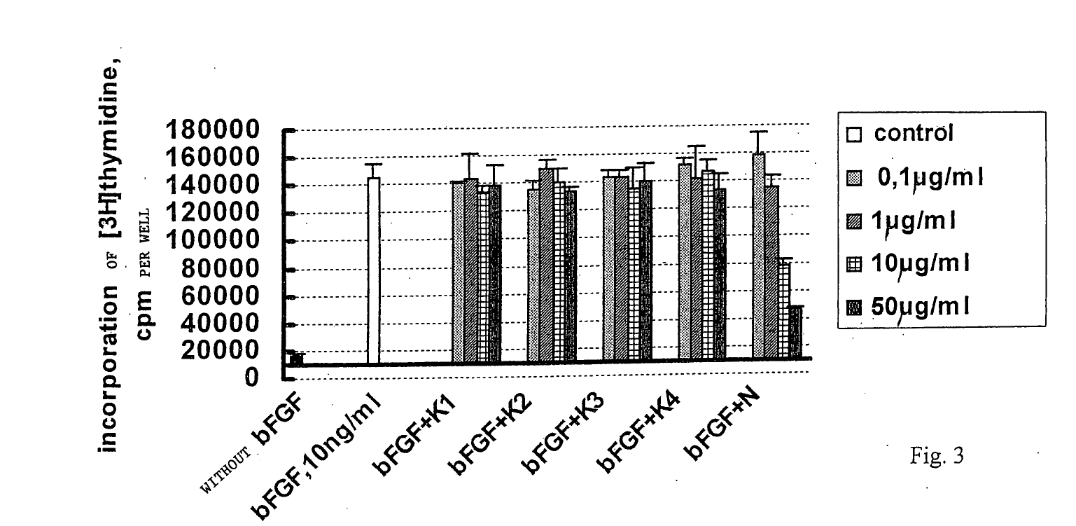

FIG. 3 represents, in the form of a graph, the results obtained during the assays relating to the stimulation of the proliferation of HUVEC cells with bFGF (10 ng/ml) in the presence of increasing doses (from 0 to 50 μg/ml) of peptide N, K1, K2, K3 or K4;

FIG. 4 is a graph illustrating the inhibition of proliferation of HUVEC cells by peptide N in the presence of a GF such as bFGF, HGF, VEGF₁₆₅ and VEGF₁₂₁;

FIG. 5 is a graph illustrating the effect of HGF, HGF+N, HGF+K1, HGF+K2, HGF+K3 and HGF+K4 on the migration of HUVEC cells through a porous membrane;

FIG. 6 comprises six photographs relating to the inhibition of the formation of a “capillary-like tube” structure by HUVECs in a three-dimensional collagen gel [comparisons of bFGF with bFGF+N, of VEGF with VEGF+N (where VEGF is VEGF₁₆₅) and of HGF with HGF+N];

FIG. 7 is a graph showing that peptide N according to the invention differs from K1, K2, K3 and K4 by virtue of the strong binding of Hep; and

FIGS. 8A-8D and 9A-9B concern complementary results obtained with said peptide N.

### ABBREVIATIONS

For convenience, the following is a list of the technical abbreviations and acronyms which have been used in the present description.
aa amino acid(s);
c-Met fragment of DNA of proto-oncogene encoding a protein, which has tyrosine kinase enzyme activity and represents the HGF receptor [herein referred to as HGF-R];
cpm counts per minute;
DNA deoxyribonucleic acid;
FBS fetal bovine serum;
FGF fibroblast growth factor; among the FGFs are distinguished in particular aFGF (acidic fibroblast growth factor) and bFGF (basic fibroblast growth factor);
GAG glycosaminoglycan;
GF growth factor;
Hep heparin;
HEPES N-2-hydroxyethylpiperazine-N-2-ethanesulfonic acid, product acting as buffer;
HGF hepatocyte growth factor; unless otherwise indicated, HGF herein denotes human hepatocyte growth factor (hHGF);
hHGF human hepatocyte growth factor;
HGF-R HGF receptor (the notation “HGF-R” is preferred herein to the nomenclature of “c-Met” which causes confusion with the “c-Met” gene);
HUVECs human umbilical cord endothelial cells;
K1 kringle-1 domain of HGF or corresponding peptide;
K2 kringle-2 domain of HGF or corresponding peptide;
K3 kringle-3 domain of HGF or corresponding peptide;
K4 kringle-4 domain of HGF or corresponding peptide;
M199 culture medium sold by the American company LIFE TECHNOLOGIES (Rockville, Md.);
M199i M199 medium supplemented with 15 mM of HEPES (pH 7.4), 2 mM of glutamine, 50 U/ml of penicillin, 50 μg/ml of streptomycin and 2.5 μg/ml of amphotericin B;
N hairpin-loop domain of HGF, the corresponding peptide or better still the peptide according to the invention whose N-terminal end is Gln (instead of PyrGlu according to the prior art illustrated by WO-A-99/55361);
NK4 peptide consisting of the amino acid sequence from N to K4 in HGF (i.e. aa 32-478 of native HGF);
RT room temperature;
RU resonance unit (1000 RU correspond to a binding of 1 ng of protein per mm²);
VEGF vascular endothelial growth factor; among the VEGFs are distinguished in particular the factors VEGF₁₆₅ and VEGF₁₂₁.

### DETAILED DESCRIPTION OF THE INVENTION

The factor hHGF has already been sequenced. In particular, the sequence PyrGlu³²-V⁴⁷⁸ of NK4 is given in WO-A-99/55361. According to the prior art, NK4 acts as an antitumor and anti-angiogenic substance according to a mechanism of competition between NK4 and HGF with respect to HGF-R, which is the receptor for HGF.

After synthesis (by expression according to a genetic engineering technique) and then purification, peptide N according to the invention, in which the first aa of the N-terminal end is Gln instead of PyrGlu) and its variants were tested. Peptide N, which is novel with respect to the teaching of the sequence of NK4, and its variants differ from K1, K2, K3 and K4 by virtue of their interactions with GAGs [in particular Hep (high molecular weight heparin, normal heparin and low molecular weight heparin), dermatan sulfate, heparan sulfate, etc.]. Overall, it is noted that N and its variants inhibit the interaction of HGF, (and of certain other GFs such as VEGF and bFGF) with GAGs; consequently, (i) they prevent the binding of the GF concerned to its receptor, and (ii) the activity of said GF is decreased or even eliminated.

It is presumed, but this is a theory by which the applicant is not bound, that said peptide N and its variants bind GAGs, which prevents:
(a) the adhesion of the GAGs to HGF or the activation of HGF by the GAGs, then
(b) the binding of HGF to HGF-R in order to activate the latter.

The result is that, in the absence of the binding of point (b) above, HGF does not exert its angiogenic effects.

It is also noted that inhibition of the mechanism (a)+(b) involves not only HGF, but also other GFs which cannot bind to HGF-R, the receptor for HGF, nor to the receptors specific for the other GFs.

The variants according to the invention are those which are derived from peptide N by deletion, substitution or addition of one or more aa.

Here, the deletion or elimination does not involve the Cys residue. On the other hand, the deletion or elimination can relate to a group of 1 to 5 contiguous aa. For example, it is possible to discard, firstly, one or more of the Met, Trp and/or Tyr residues and, secondly, a peptide fragment of no more than 5 aa located in the sequence of N, in particular between (a) Val³³ and Glu³⁸, (b) Leu⁶⁶ and Val⁷⁷, or (c) Glu⁸⁴ and Asn⁹⁰.

The substitutions here relate to one or more groups of 1 to 3 aa. For example, at least one of the Cys (or even all of them) can be replaced with Ser, and certain residues with a basic side chain (in particular Arg, His and Lys) can be replaced with Ala, Glu or Asp.

The additions involve mainly fragments of 1 to 3 aa with a basic side chain (preferably Arg and/or Lys) close to the isolated basic amino acids of N, in particular close to Lys¹⁶, Lys²¹, Arg⁴², Arg⁴⁵, Lys⁴⁷, Lys⁵⁴, Lys⁶⁰, His⁸³, Lys⁹¹ and Arg⁹⁵. It is also possible, without modifying the activity of N, to incorporate 1 to 22 aa upstream of Gln¹ (i.e. upstream of Gln³² in the sequence of native HGF), according to the expression vector used. Thus, it is possible to add, upstream of Gln¹, one of the following N-terminal ends:
(A) M,
(A2) MASMTGGQQMGRD,
(A3) MGSSHHHHHSSGLVPRGSHM.

The variants of peptide N according to the invention may, of course, result from a combination of deletions, substitutions and/or additions.

The method of preparation which is advocated according to the invention consists in using a DNA molecule encoding peptide N or one of its variants, and then
(1) introducing on the 5′-end side a DNA fragment encoding a first restriction site,
(2) introducing on the 3′-end side a DNA fragment encoding a second restriction site, steps 1 and 2 being carried out in any order, and then
(3) inserting the peptide resulting from steps 1 and 2 together into a bacterial plasmid, in particular a plasmid of Escherichia coli,
(4) culturing the strain containing the plasmid thus modified at a temperature of 30-37° C.,
(5) expressing peptide N or one of its variants at a temperature of 25-30° C.

The peptide thus obtained is purified according to a method known per se, in particular by chromatography.

### EXAMPLES 1 to 4

Example 1 of the invention consists of peptide N, the aa sequence of which is given in SEQ ID No.: 2. The DNA fragment expressing Ex 1 is described in SEQ ID No.: 1.

Example 2 of the invention is the peptide consisting of A3-N. It comprises peptide A3 at its N-terminal end upstream of peptide N. The aa sequence of Ex 2 is given in SEQ ID No.: 4. The DNA fragment expressing Ex 2 is provided in SEQ ID No.: 3.

Example 3 of the invention results from the deletion of a fragment of 4 amino acids (aa Met⁹⁴ to Gly⁹⁷) from Ex 2 (i.e. Met⁷³ to Gly⁷⁶ of peptide N). The sequence of Ex 3 and that of the DNA which encodes it are given in SEQ ID No.: 5 and SEQ ID No.: 7, respectively.

Example 4 of the invention results from Ex 2, firstly, by insertion of 2 aa (Lys-Arg) after Lys¹¹² (i.e. Lys⁹¹ of N) and, secondly, replacement of the second and third Cys residues [Cys⁶⁴ and Cys⁷⁴ of Ex 2 (i.e. Cys⁴³ and CyS₅₃ of N)] with Ser. The sequence of Ex 4 and that of the DNA which encodes it are given in SEQ ID No.: 6 and SEQ ID No.: 8, respectively.

Some of the results of the assays which were carried out have been summarized hereinafter. In these assays, the HUVEC cells were used as principal model for analyzing angiogenesis ex vivo. HUVEC cells do not produce HGF; on the other hand, they provide the c-Met proto-oncogene which expresses the HGF receptor HGF-R and the level of which can be modulated according to the culture conditions. Quiescent cells, at confluence, provide a low amount of HGF-R. This expression increases when the cells enter the exponential growth phase, or when they form capillary-like structures (or capillary tubes) in a three-dimensional collagen gel.

Assay 1

The stimulation of the proliferation of HUVEC cells with bFGF was assessed in the absence and in the presence of increasing amounts of N, K1, K2, K3 and K4 peptides, the HUVEC cell proliferation being measured by means of [³H] thymidine.

The HUVEC cells are collected when they have reached the confluent state and are placed on 24-well plates at a concentration of 2×10⁴ cells/well in 0.5 ml of M199i supplemented with 2.5% of FBS. The cells are left to attach for 4 h at 37° C. and proliferation is induced by means of 10 ng/ml of bFGF added alone or in combination with increasing amounts (0.1-50 μg/ml) of purified peptide N, K1, K2, K3 or K4. After incubation for a further 24 h, 3.7×10⁴ Bq (1 μCi) of [³H] thymidine (product sold by the company Amersham Pharmacia Biotech) are added to each well for the final 18 h of incubation. The cells are washed three times with PBS and then treated for 0.5 h with a 10% (w/v) solution of trichloroacetic acid cooled in a bath of ice. The resulting precipitates are solubilized with 0.3 N NaOH (0.5 ml/well) for 0.5 h at RT. The solutions are then neutralized with 1N HCl. The radioactivity [³H] is measured by means of a liquid scintillation counter (Beckman LS-6500 multipurpose scintillation counter) . The results are expressed in the form of the mean [count/minute (± standard error of the mean)] of three cultures per dose of test product.

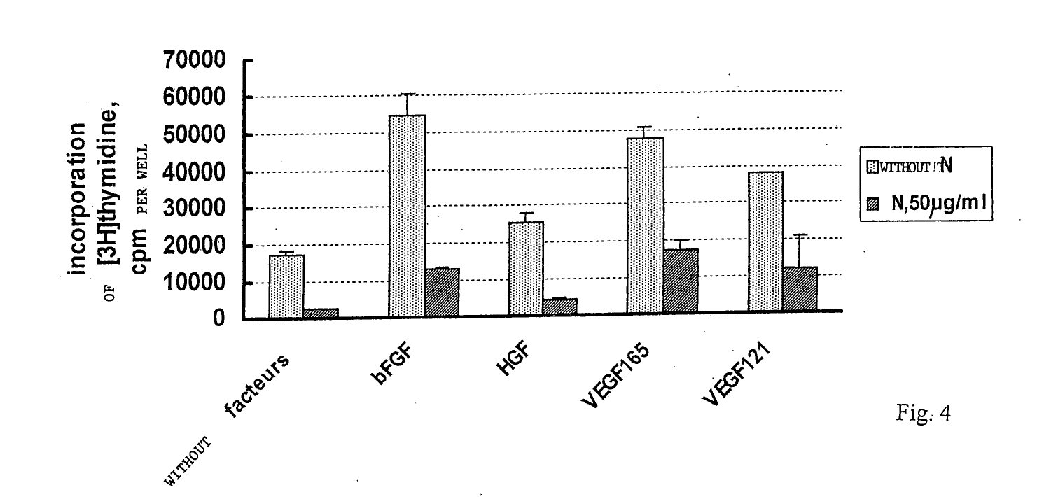

The results recorded in FIG. 3 show that none of the kringles K1 to K4 has an effect on HUVEC cell proliferation induced by bFGF since the [³H] thymidine count does not vary in a statistically significant manner. On the other hand, peptide N inhibits the HUVEC cell proliferation induced by bFGF according to a concentration-dependent mode, half the maximum effect occurring at a dose of peptide N of approximately 7 μM.

Assay 2

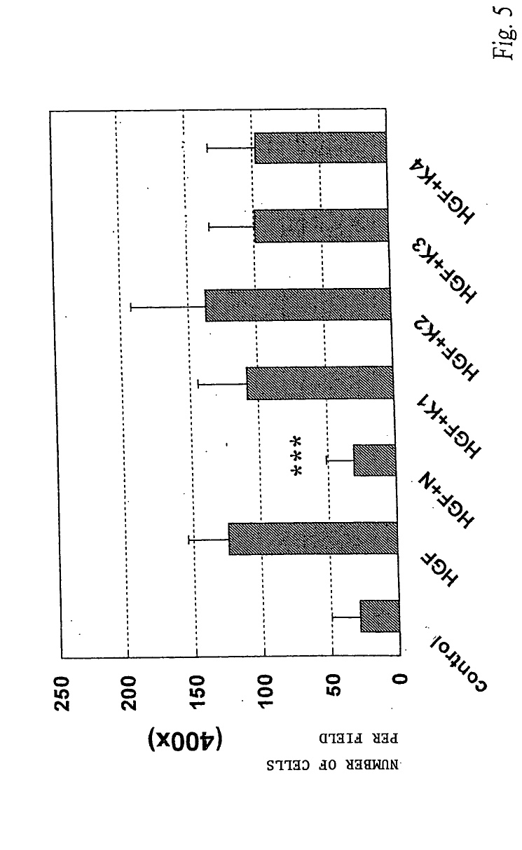

The proliferation of HUVEC cells was assessed according to the modalities described above, inducing the proliferation of the cells by adding 10 ng/ml of HGF, or 10 ng/ml of each of the isoforms of VEGF (VEGF₁₆₅ and VEGF₁₂₁) with bFGF to the wells, in the absence or in the presence of 4 μM of peptide N. The results recorded in FIG. 4 (which are expressed in cpm) show that peptide N notably inhibits the HUVEC cell proliferation induced by any one of the GFs.

Assay 3

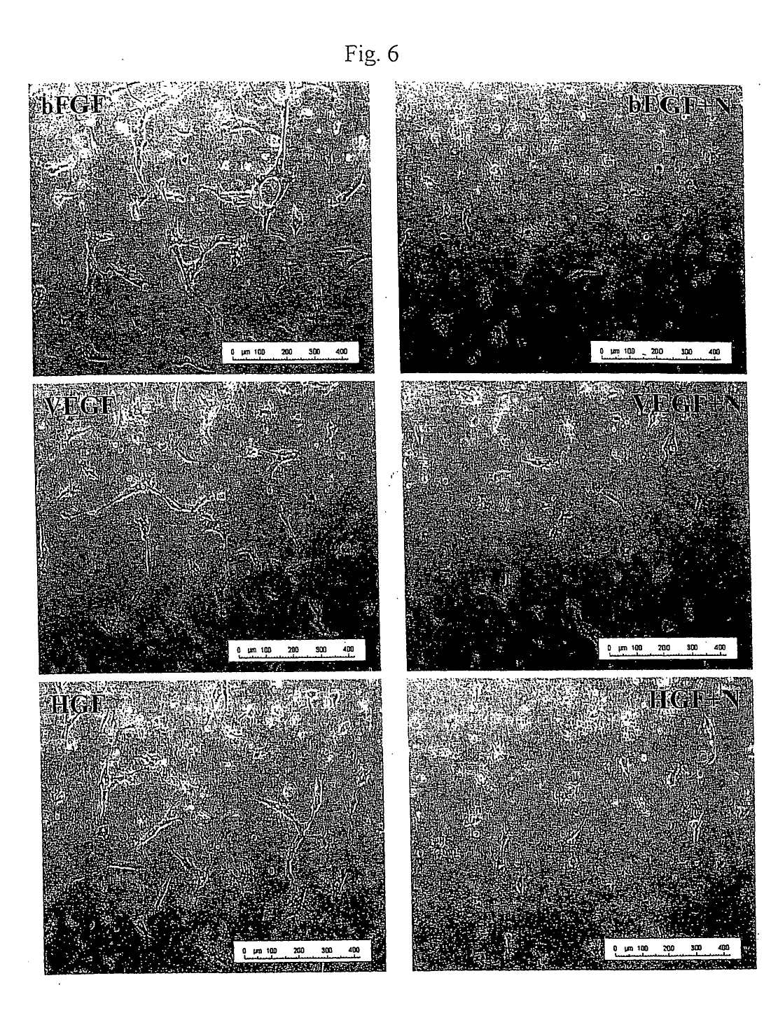

The chemotactic migration of HUVEC cells was evaluated according to a technique derived from the prior art (modified assay with Boyden chamber). Parietal cell culture chamber (“Transwell” ) inserts sold by the company Corning Costar Corporation (Cambridge, Mass.) and equipped with porous polycarbonate filters (pore dimensions: 8 μm) were coated with 0.1% of gelatin. HUVEC cells (4×10⁴) in M199i medium supplemented with 2.5% of FBS were added to the Transwell devices. The inserts are placed over chambers containing 10 ng/ml of HGF and one of the purified peptides N, K1, K2, K3 or K4, each at a dose of 50 μg/ml. The chambers are incubated for 5 h at 37° C. The filters are then rinsed with PBS, fixed and stained with hematoxylin. The upper surface of the filters is scraped in order to remove the cells which have not migrated. The number of cells in a field of high magnification (×400) is recorded, at a rate of 10 fields/filter, in triplicate. The results recorded in FIG. 5 demonstrate that peptide N completely inhibits the induced migration of the HUVEC cells through the porous membrane. It is also observed that some kringles (K3 and K4) exhibit a weak inhibitory effect, this effect not, however, being statistically significant.

Assay 4

Inhibition of the formation of HUVEC cell capillary tubes in a three-dimensional collagen gel, by peptide N, was studied. Type 1 rat tail collagen was obtained from the company BD Biosciences (Bedford, Mass.) and the gels were formed according to the manufacturer's recommendations. Briefly, an ice-cold solution of collagen is neutralized by adding 1N NaOH and mixed with PBS in a 9/1 v/v ratio. HUVEC cells, isolated by treatment with trypsin-EDTA and suspended in M199i medium supplemented with 2.5% of FBS, are mixed with the neutralized solution of collagen so as to obtain a final concentration of 1 mg/ml of collagen. 0.4 ml of this suspension (5×10⁵ cells/ml) is poured into the wells of a 24-well plate and the gels are allowed to form by incubation at 37° C. for 40 minutes. The gels are covered with 0.5 ml of M199i medium supplemented with 2.5% of FBS and 10 ng/ml of one of the three GFs: bGFG, VEGF₁₆₅ or HGF. Purified peptide N (1 μM) is added to wells at the same time as the growth factor. The cultures are incubated for 48 h at 37° C. under a humid atmosphere containing 5% CO₂. The gels are examined using a microscope (“Zeiss Axiovert 25”) and the images are recorded using the “Archimed Pro 2.1” software.

When HUVEC cells are grown in a collagen gel, they rapidly undergo a morphogenesis to give a network of capillary tubes.

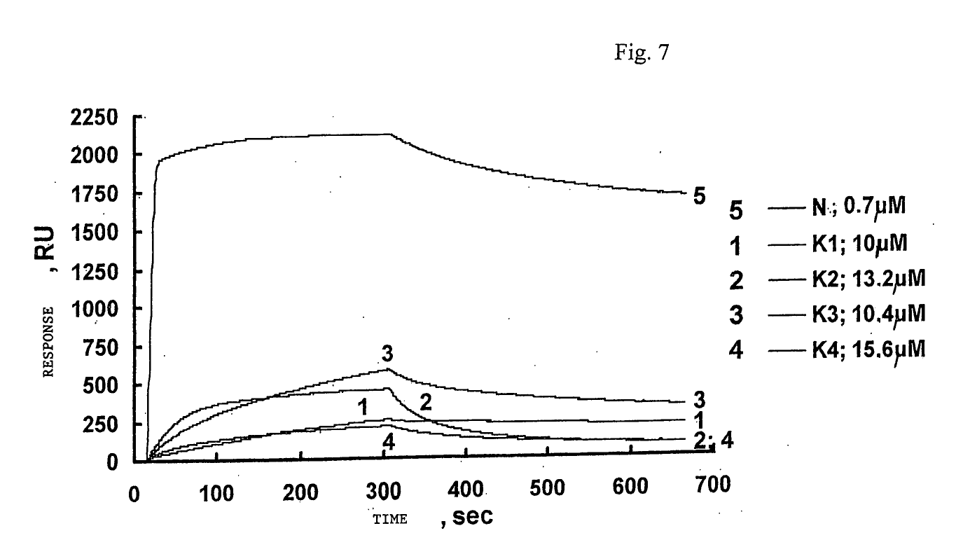

The formation of these tubes occurs spontaneously or is induced by growth factors. The results recorded in FIG. 6 show that bFGF, VEGF₁₆₅ and HGF induce, under the experimental conditions used, organization of most of the HUVEC cells into capillary tubes. The formation of these tubes is completely inhibited by peptide N according to the invention, when it is added at the same time as any one of said growth factors. In addition, no modification of the morphogenesis of the HUVEC cells is observed when capillary tube formation is induced in the presence of one of the kringles K1, K2, K3 or K4 (these results do not appear in FIG. 6).

Assay 5

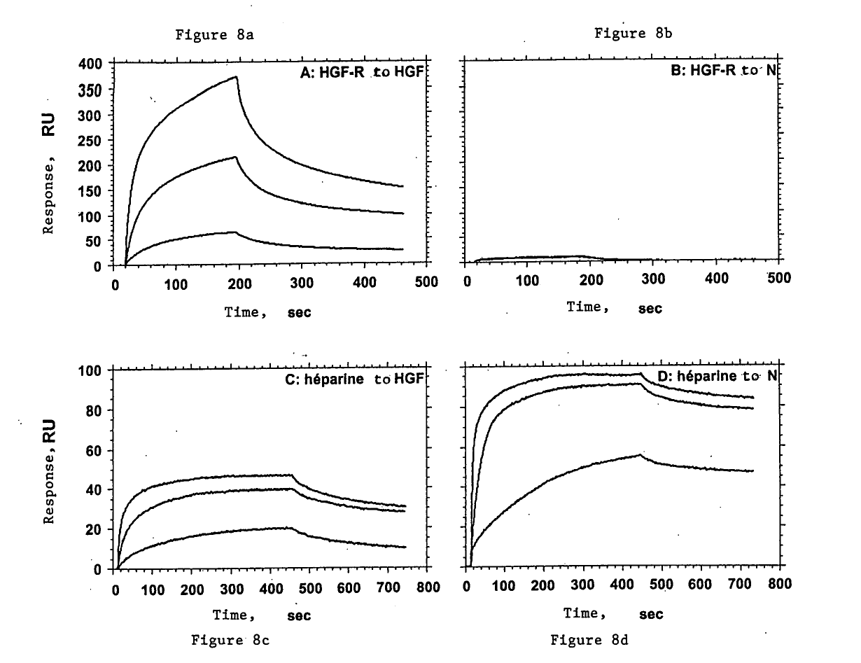

In order to demonstrate that peptide N according to the invention constitutes the main site involved in the interaction of HGF with a GAG such as Hep, the affinities of peptides N, K1, K2, K3 and K4 for heparin were analyzed by surface plasmon resonance using a “BIAcore 2000” instrument (sold by the company BIAcore, Uppsala, Sweden). High molecular weight heparin was biotinylated with the product EZ-Link Biotin-LC-Hydrazide (manufactured by the company Pierce, Rockford, Ill.) and was used so as to be captured on the surface of a detector (“sensor ship”) coated with streptavidin. The purified peptides were diluted in the usual buffer for the BIAcore instrument (PBS containing 0.005% of tween-20) and injected at a rate of 20 ml/minute onto immobilized heparin. The experiments were carried out within a peptide concentration range. The results obtained were analyzed automatically by means of the BIAevaluation program from the company BIAcore. The results obtained were recorded in FIG. 7, where it is seen that peptide N has a very high affinity for heparin compared to the other four kringles K1, K2, K3 and K4. The five peptides are capable of binding to heparin, but they exhibit differences in the binding. The greatest response is that obtained for the binding of 0.7 μM of peptide N, i.e. a concentration approximately 20 times lower than those of the kringles (about 2000 RU for peptide N instead of 600 RU for K3). At physiological concentrations, the HGF/GAG interaction is therefore mainly provided by peptide N.

Assay 6

Assay 5 above was reproduced in order to assess the affinity of the binding of soluble HGF-R [chimeric product which is a fusion protein (HGF-R/IgG) comprising the HGF-binding portion of the extracellular domain of HGF-R and a portion of IgG], of normal molecular weight heparin (Hep) and of fractionated low molecular weight heparin (LMWH) to HGF or N, each one immobilized on the surface of the detector (“sensor ship”).

It is noted that Hep has a higher affinity (25 times higher) than LMWH with respect to HGF and N.

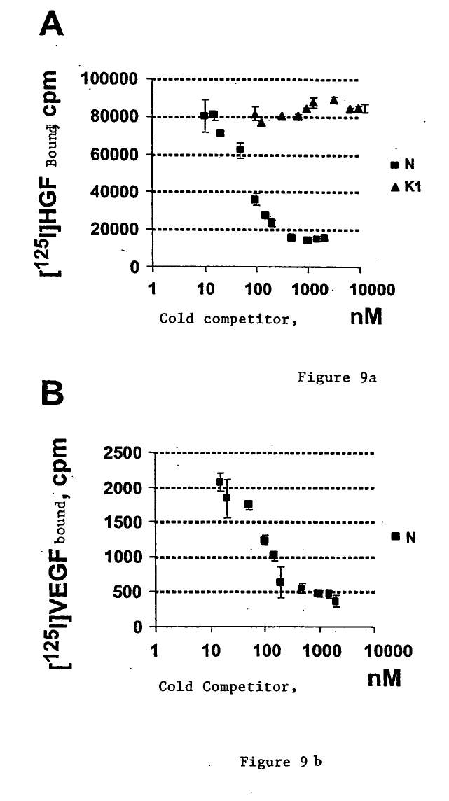

As regards the binding of soluble HGF-R and Hep to HGF and N, the results obtained, which are recorded in FIGS. 8A, 8B, 8C and 8D for three different doses, demonstrate the selective binding of Hep to N and the absence of binding of soluble HGF-R to N.

Assay 7

Radiolabeled ligand binding and displacement was studied. HGF (5 μg), VEGF₁₆₅ (10 μg) and N (25 μg) (or, where appropriate, K1 (25 μg)] were iodinated with 1 mCi (i.e. 3.7×10⁷ Bq) of [¹²⁵I]Na using “Iodo-gen Pre-Coated Iodination Tubes” according to the manufacturer's instructions. The isolated proteins were purified on columns equilibrated with 25 mM of Tris-HCl at pH 7.4, containing 0.4 M of NaCl, 5 mM of EDTA and 0.25% of AlbuMAX (provided by the company INVITROGEN), and concentrated by centrifugation. The specific activities determined by precipitation with trichloroacetic acid are:
18×10⁶ cpm/pmol for [¹²⁵I]HGF,
8×10⁶ cpm/pmol for [¹²⁵I]VEGF₁₆₅, and
9×10⁵ cpm/pmol for [¹²⁵I]N.

The binding and displacement studies with the iodinated proteins were carried out on HUVEC cells at confluence, at 4° C., on 24-well plates. The cells were washed with PBS and preincubated for 0.5 h with 0.5 ml of binding medium (normal medium containing 0.1% of AlbuMAX). To determine the binding parameters with regard to N, increasing amounts of radiolabeled N were added to the HUVEC cells, with or without a molar excess of unlabeled N of 100, and incubated for 2 h. For the displacement experiment, constant amounts of HGF (1 nM) or of VEGF₁₆₅ (0.2 nM) were added to the HUVEC cells in the presence of an increasing concentration of unlabeled N, for 4 h and 2 h, respectively. At the end of incubation, the cells were washed three times with the binding medium and then solubilized with 0.3 M of NaOH. The radioactivity associated with the HUVEC cells was determined using an appropriate counter.

The results obtained show that N inhibits the binding of HGF and VEGF₁₆₅ to HUVECs, and that K1 does not act here as an inhibitor. The inhibition by N depends on the concentration of said N.

As regards the displacement, it is observed that the kinetics correspond to a simple reaction model of bimolecular type. The Kd values calculated using an appropriate program are
20.40±5.11 nM for HGF, and
56.17±17.18 nM for VEGF₁₆₅.

See FIGS. 9A and 9B.

Conclusions

In view of the results given above in assays 1-7 and recorded in FIGS. 3-9, it is clear that peptide N acts as an HGF inhibitor. Unlike the already known peptide NK4, peptide N according to the invention does not substantially intervene on the competitive binding with HGF to the HGF-R receptor. On the other hand, in view of assays 5-6 and of FIGS. 7-8, peptide N intervenes more precisely on the interaction of HGF with a GAG such as Hep by preventing the adhesion of Hep to HGF or the activation of HGF by Hep; consequently, HGF can no longer act on or activate HGF-R so that the latter exerts its tyrosine kinase effect.

The peptides of examples 2-4 exhibit the same mode of action as peptide N. As anti-angiogenic substances, N and its variants are useful in the treatment of disorders related to angiogenesis and in particular induced by hyperangiogenesis. They are particularly indicated
as neovascularization inhibitors and HGF antagonists (in particular with respect to rheumatoid arthritis, to diabetic retinopathy, to age-related macular degeneration and to excessive scar formation during wound healing),
as modulators of abnormal cell proliferation (in particular with respect to psoriasis, to

Osler-Webber syndrome, to angiomas and to benign or malignant tumors), and
as inhibitors of endothelial cell hyper-stimulation.

Briefly, peptide N and its variants are particularly advantageous as anti-neovascularization, anti-rheumatic, anti-arthritic, anti-inflammatory, anti-psoriatic, anti-proliferative, antitumor and/or cytostatic agents.

1. The use of a peptide substance, characterized in that use is made of a peptide substance selected from the group consisting of:
(i) peptide N derived from the HGF α-chain and having the amino acid sequence represented by SEQ ID No.: 2, and
(ii) its peptide variants derived from said peptide N sequence by deletion, substitution or addition of one or more amino acids,
said peptide N and its variants having an anti-angiogenic activity in accordance with a mechanism different from competitive binding of HGF to the HGF-R receptor,
said peptide N and its variants selectively binding GAGs such as heparin, and preventing the interaction of HGF with GAGs by virtue of which HGF binds to or activates the HGF-R receptor,
for preparing an anti-angiogenesis medicinal product intended for therapeutic use with respect to angiogenesis-related disorders.

2. A therapeutic composition, characterized in that it contains, in combination with a pharmaceutically acceptable excipient, a therapeutically effective amount of a peptide substance selected from the group consisting of:
(i) peptide N derived from the HGF α-chain and having the amino acid sequence represented by SEQ ID No.: 2, and
(ii) its peptide variants derived from said peptide N sequence by deletion, substitution or addition of one or more amino acids,
for combating angiogenesis-related disorders.

3. A peptide substance belongin g to the group comprising:
(i) peptide N of the HGF α-chain, and
(ii) the variants which are derived therefrom by deletion, substitution or addition of one or more amino acids,
said peptide substance being characterized in that it is selected from the peptides represented by SEQ ID No.: 2, SEQ ID No.: 4, SEQ ID No.: 5 and SEQ ID No.: 6.

4. A DNA fragment, characterized in that it encodes the peptide substance as claimed in claim 3.

5. A plasmid, characterized in that it contains the DNA fragment as claimed in claim 4.

6. A bacterial strain containing the plasmid as claimed in claim 5.

7. A method for preparing a peptide substance as claimed in claim 3, in which use is made of a DNA molecule encoding peptide N or one of its variants, said method being characterized in that it comprises the steps consisting in:
(1) introducing on the 5′-end side a DNA fragment encoding a first restriction site,
(2) introducing on the 3′-end side a DNA fragment encoding a second restriction site, steps 1 and 2 being carried out in any order, and then
(3) inserting the peptide resulting from steps 1 and 2 and together into a bacterial plasmid, in particular a plasmid of Escherichia coli,
(4) culturing the strain containing the plasmid thus modified at a temperature of 30-37° C., and
(5) expressing peptide N or one of its variants at a temperature of 25-30° C.

8. The peptide substance as claimed in claim 3, characterized in that its amino acid sequence is that of SEQ ID No.: 4.

9. The peptide substance as claimed in claim 3, characterized in that its amino acid sequence is that of SEQ ID No.: 5.

10. The peptide substance as claimed in claim 3, characterized in that its amino acid sequence is that of SEQ ID No.: 6.

11. The peptide substance as claimed in claim 3, characterized in that its amino acid sequence is that of SEQ ID No.: 2.


SEQUENCE LISTING (8 sequences)

SEQ ID NO: 1 - DNA, 288 bp, HUMAN
  CDS (1)..(288)
caaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaac

SEQ ID NO: 2 - protein, 96 aa, HUMAN
QRKRRNTIHEFKKSAKTTLIKIDPALKIKTKKVNTADQCANRCTRNKGLPFTCKAFVFDKARKQCLWFPFNSMSSGVKKEFGHEFDLYENKDYIRN

SEQ ID NO: 3 - DNA, 351 bp, Artificial Sequence
  CDS (1)..(351): DNA encoding peptide A3 upstream of peptide N
atgggcagcagccatcatcatcatcatcacagcagcggcctggtgccgcgcggcagccatatgcaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaac

SEQ ID NO: 4 - protein, 117 aa, Artificial Sequence
  Peptide A3 upstream of peptide N
MGSSHHHHHHSSGLVPRGSHMQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRN

SEQ ID NO: 5 - protein, 113 aa, Artificial Sequence
  Modification of SEQ ID NO. 4 peptide to delete Met94 through Gly97
MGSSHHHHHHSSGLVPRGSHMQRKRRNTIHEFKKSAKTTLIKIDPALKIKTKKVNTADQCANRCTRNKGLPFTCKAFVFDKARKQCLWFPFNSVKKEFGHEFDLYENKDYIRN

SEQ ID NO: 6 - protein, 119 aa, Artificial Sequence
  Modification of SEQ ID NO. 4 peptide to insert Lys-Arg after Lys112, and replacement of Cys64 and Cys74 with Ser
MGSSHHHHHHSSGLVPRGSHMQRKRRNTIHEFKKSAKTTLIKIDPALKIKTKKVNTADQCANRSTRNKGLPFTSKAFVFDKARKQCLWFPFNSMSSGVKKEFGHEFDLYENKKRDYIRN

SEQ ID NO: 7 - DNA, 339 bp, Artificial Sequence
  CDS (1)..(339): DNA encoding SEQ ID NO. 5 peptide
atgggcagcagccatcatcatcatcatcacagcagcggcctggtgccgcgcggcagccatatgcaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcgtgaaaaaagaatttggccatgaatttgacctctatgaaaacaaagactacattagaaac

SEQ ID NO: 8 - DNA, 357 bp, Artificial Sequence
  CDS (1)..(357): DNA encoding SEQ ID NO. 6 peptide
atgggcagcagccatcatcatcatcatcacagcagcggcctggtgccgcgcggcagccatatgcaaaggaaaagaagaaatacaattcatgaattcaaaaaatcagcaaagactaccctaatcaaaatagatccagcactgaagataaaaaccaaaaaagtgaatactgcagaccaatgtgctaatagaagcactaggaataaaggacttccattcactagcaaggcttttgtttttgataaagcaagaaaacaatgcctctggttccccttcaatagcatgtcaagtggagtgaaaaaagaatttggccatgaatttgacctctatgaaaacaaaaaaagggactacattagaaac